SEQUENCE LISTING (22 sequences)

SEQ ID NO: 1 - protein, 11 aa, Artificial Sequence
  Description of Artificial Sequence: Synthetic peptide
CDPGYIGSRKK

SEQ ID NO: 2 - protein, 14 aa, Artificial Sequence
  Description of Artificial Sequence: Synthetic peptide
LRGEVGLPGVKGDK

SEQ ID NO: 3 - protein, 14 aa, Artificial Sequence
  Description of Artificial Sequence: Synthetic peptide
GPKGDRGFPGTPGI

SEQ ID NO: 4 - protein, 8 aa, Artificial Sequence
  Description of Artificial Sequence: Synthetic peptide
GRGDSPKK

SEQ ID NO: 5 - protein, 9 aa, Artificial Sequence
  Description of Artificial Sequence: Synthetic peptide
KKVAAKSGG

SEQ ID NO: 6 - protein, 10 aa, Artificial Sequence
  Description of Artificial Sequence: Synthetic peptide
GRGDSPHSRN

SEQ ID NO: 7 - protein, 6 aa, Artificial Sequence
  Description of Artificial Sequence: Synthetic peptide
RRRRRR

SEQ ID NO: 8 - protein, 12 aa, Artificial Sequence
  Description of Artificial Sequence: Synthetic peptide
  MOD_RES (9)..(9): DOPA
AKPSYPPTXKKK

SEQ ID NO: 9-10 - skipped (2 entries)

SEQ ID NO: 11 - protein, 6 aa, Artificial Sequence
  Description of Artificial Sequence: Synthetic peptide
  MOD_RES (1)..(1): D-Phe
FGGGGG

SEQ ID NO: 12 - protein, 6 aa, Artificial Sequence
  Description of Artificial Sequence: Synthetic peptide
  MOD_RES (1)..(1): D-Phe
FFGGGG

SEQ ID NO: 13 - protein, 6 aa, Artificial Sequence
  Description of Artificial Sequence: Synthetic peptide
  MOD_RES (1)..(1): D-Phe
FFFGGG

SEQ ID NO: 14 - protein, 6 aa, Artificial Sequence
  Description of Artificial Sequence: Synthetic peptide
  MOD_RES (1)..(1): D-Phe
FFFFGG

SEQ ID NO: 15 - protein, 6 aa, Artificial Sequence
  Description of Artificial Sequence: Synthetic peptide
  MOD_RES (1)..(1): D-Phe
FFFFFG

SEQ ID NO: 16 - protein, 198 aa, Artificial Sequence
  Description of Artificial Sequence: Synthetic polypeptide
  misc_feature (1)..(99): This region may encompass 1-99 residues wherein the entire sequence may not be longer than 100 residues in length
  misc_feature (100)..(198): This region may encompass 1-99 residues wherein the entire sequence may not be longer than 100 residues in length
FFFFFFFFFFFFFFFFFFFFFFFFFFFFFFFFFFFFFFFFFFFFFFFFFFFFFFFFFFFFFFFFFFFFFFFFFFFFFFFFFFFFFFFFFFFFFFFFFFFGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGG

SEQ ID NO: 17 - protein, 20 aa, Artificial Sequence
  Description of Artificial Sequence: Synthetic peptide
PPPPPPPPPPPPPPPPPPPP

SEQ ID NO: 18 - protein, 23 aa, Artificial Sequence
  Description of Artificial Sequence: Synthetic peptide
WLRPPPPPPPPPPPPPPPPPPPP

SEQ ID NO: 19 - protein, 11 aa, Artificial Sequence
  Description of Artificial Sequence: Synthetic peptide
ACSSSPSKHCG

SEQ ID NO: 20 - protein, 11 aa, Artificial Sequence
  Description of Artificial Sequence: Synthetic peptide
YARAAARQARA

SEQ ID NO: 21 - protein, 21 aa, Artificial Sequence
  Description of Artificial Sequence: Synthetic peptide
YLRRIKAYLRRIKAYLRRIKA

SEQ ID NO: 22 - protein, 20 aa, Artificial Sequence
  Description of Artificial Sequence: Synthetic peptide
  misc_feature (1)..(20): This sequence may encompass 2-20 residues
RRRRRRRRRRRRRRRRRRRR

(19) United States
(12) Patent Application Publication
(10) Pub. No.: US 2013/0224269 A1
(43) Pub. Date: Aug. 29, 2013

(54) Biofunctional Anchored Extended-Wear Cosmetics

(71) Applicants: Raheel Khan, Franklin Park, NJ (US); Amitabh Bansal, Hoboken, NJ (US); Siming W. Chen, Basking Ridge, NJ (US); Uma Santhanam, Tenafly, NJ (US); Hussam H. Shaheen, Lebanon, NH (US)

(72) Inventors: Raheel Khan, Franklin Park, NJ (US); Amitabh Bansal, Hoboken, NJ (US); Siming W. Chen, Basking Ridge, NJ (US); Uma Santhanam, Tenafly, NJ (US); Hussam H. Shaheen, Lebanon, NH (US)

(73) Assignee: AVON PRODUCTS, INC., Suffern, NY (US)

(21) Appl. No.: 13/817,570

(22) PCT Filed: Nov. 3, 2011
(86) PCT No.: PCT/US11/59051

§ 371 Date: Feb. 19, 2013

Related U.S. Application Data
(60) Provisional application No. 61/413,729, filed on Nov. 15, 2010.

(51) Int. Cl. A61K 8/02; A61Q 1/02

(52) U.S. Cl. CPC A61K 8/0245; A61Q 1/02
USPC 424/401

(57) ABSTRACT

An extended wear cosmetic composition for application to a keratinous surface comprising a cosmetic particle coated with a biofunctional polymer wherein said coated particle adheres to the keratinous surface for an extended time period. A delivery system for conjugating a biofunctional polymer and cosmetic particle to specific cells on a keratinous surface such that the particle adheres to the keratinous surface for an extended time period.

### CROSS REFERENCE TO RELATED APPLICATIONS

This application claims priority to International Application Serial No. PCT/US11/59051 filed Nov. 3, 2011, which claims priority U.S. Provisional Patent Application No. 61/413,729, filed Nov. 15, 2010, the contents of which application are hereby incorporated by reference in their entirety.

### FIELD OF THE INVENTION

The present invention relates generally to cosmetic particles that are anchored on a keratinous surface such as skin, hair, and nails to exhibit long wear and reduced transference.

### BACKGROUND OF THE INVENTION

Women are continually seeking cosmetic products that maintain a fresh look for extended periods of time and that are substantially transfer resistant. A major problem with the current cosmetics, skin care and hair care compositions, as well as nail coloring agents is that they lack the required durability required for long-lasting effects unless they contain harsh solvents and/or specialty polymers that affect skin feel. A popular trend is “permanent make-up” which employs a form of tattooing (permanent pigmentation of the dermis) as a means of producing designs that resemble makeup, such as eyelining and other permanent enhancing colors to the skin of the face, lips, and eyelids. As the name suggests, however, this practice is permanent. Oftentimes, women simply desire a long-lasting or extended-wear cosmetic which naturally fades away or can be removed manually. Currently, long wear and/or waterproof attributes of cosmetics are achieved by specialty polymers and/or waxes, and/or harsh solvents or specialty polymers, which usually allow for a wear of only 12 or so hours. These current long wearing cosmetics typically lose their luxuriousness and silky/soft feel because of the use of these specialty polymers and waxes. Additionally, current long wear cosmetics require the use of abrasives, chemical removal agents and/or extensive washing.

The use of biofunctional polymers, also known as polymeric biomaterials, is well known in connection with drug delivery and therapeutic applications—such as, for example, contrast agents for MRI. Pharmacologically active polymers are another class of biofunctional materials, with the goal of being used for prolonged, site-specific pharmacotherapy. Typically, the drug particles are extremely small (nm range) because they have to pass through various membranes within the body. Additionally, the biofunctional polymers are typically of a very specific nature to facilitate binding to specific cells. The biofunctional polymers used in drug delivery and therapeutic applications usually comprise ingredients such as specific peptides to target specific biomolecules.

Cosmetic products comprising biofunctional polymers that are chemically bonded or otherwise physically connected or coupled with cosmetic ingredients such as colorants, pigments and other active agents that will adhere to specific surfaces of the skin, hair or nails for extended lengths of time are highly desirable. As described herein, novel and beneficial methods and compositions, as well as their mode of action, for extended wear cosmetics are provided.

### SUMMARY OF THE INVENTION

The invention provides cosmetic products that bind with high affinity to keratinous surfaces such as hair, skin including lips and nails.

It is an object of the present disclosure to provide a cosmetic delivery system that facilitates the adherence of a cosmetic particle composition to a keratinous substrate or surface, namely skin, nails and hair, and which remains on the keratinous surface for long or extended lengths of time.

In one aspect of the invention, a cosmetic product is provided comprising particles (e.g., colorants, powders, biological agents and the like) that are attached with a biofunctional polymer that facilitates the adherence of the particles to cells on a keratinous surface.

In another aspect, the invention provides a delivery system that comprises a specific biofunctional polymer that is attached to a particle and which, when applied on a keratinous surface, has affinity for binding with specific desired cells on the keratinous surface.

The foregoing discussion is presented solely to provide a better understanding of the nature of the problems confronting the art and should not be construed in any way as an admission as to prior art nor should the citation of any reference herein be construed as an admission that such reference constitutes “prior art” to the instant application.

### BRIEF DESCRIPTION OF THE FIGURES

FIG. 1 schematically illustrates a biofunctional polymer unit of the present invention useful for conjugation to a particle.

FIG. 2 schematically illustrates a particle-biofunctional polymer conjugate of the present invention adhered to a biological surface.

### DETAILED DESCRIPTION

All terms used herein are intended to have their ordinary meaning unless otherwise provided.

The present disclosure provides particles, particularly colorants and pigments, and optical agents that are attached with a biofunctional polymer having a high affinity for desired keratinous surface cells and which facilitate the adherence of the colorant and pigment particles to the keratinous surface such as the skin, hair or nails.

As used herein, the term “attached” and/or “attaching” are intended to connote at least some degree of surface adherence of the biofunctional polymer to the particles via chemical or physical bonds. Illustrative examples include silane coupling, covalent bonds, avidin-biotin conjugated complexes, hydrogen bonds, and the like, of the biofunctional polymer to the cosmetic active, hereinafter described in terms of a particle having a cosmetic benefit, for example a particle that provides coloration to the skin or a change in the optical properties of the skin. It is not required that a particle be completely surrounded or enveloped by the biofunctional polymer. The application of an amount of biofunctional polymer on the particle is intended to render the particle as “attached” or having an “attachment”. It is also intended that the “attachment” include wholly or partially covering or coating a particle with a material that forms a thin continuous layer over that portion of the substrate to which it is applied, and could be referred to as a film.

Throughout this disclosure the cosmetic particle, active agent and/or optical agent attached to the biofunctional polymer may be referred to as the “polymer-particle conjugate.”

The biofunctional polymer serves multiple functions. In one aspect, the selection of a specific biofunctional polymer can be used to target specific cells, biomolecules or targets on the skin surface to achieve a benefit, e.g., hiding a wrinkle or covering a blemish that is physiologically and/or biologically different than a regular skin cell. In another aspect, the biofunctional polymer is selected to provide a specific and/or desired length of time for adhering the particle to the skin or other keratinous surface. The biofunctional polymer typically provides for long/extended wear of the product and renders the applied pigment/colorant/optical agent transfer resistant.

In contrast to pharmaceutical particles that must be small enough to pass through various biological barriers, the cosmetic particles according to the present invention preferably have a median particle size in the range of approximately 500 nm-50 microns. More preferably the cosmetic particle has a median particle size in the range of approximately 5-15 microns and most preferably is about 5 microns. With particles of such size, the cosmetic particle is rendered too big to be “pulled” into or through the skin with the biofunctional peptide and therefore the particle remains anchored on the skin surface to provide its intended benefit.

The peptides in the biofunctional polymer are hydrophobic and have a surface energy that closely mimics the surface energy of the stratum corneum thus making them compatible to the skin cell. Accordingly, the biofunctional polymer has a capacity to penetrate and bond with one or more skin cells upon application of the cosmetic product to the skin. Similarly, the polymer attached particles of the invention can be applied to hair or nails. This phenomenon allows for the particles to exhibit long wear because the peptide that penetrates or attaches to the cell of the skin, nail, or hair upon which it is applied also attaches the cosmetic particle to the cell of the skin, nail, or hair thereby keeping it anchored and sitting on the keratinous surface. As stated above, the length of wear will depend on the biofunctional polymer and can be selected to either remain anchored on the surface until the cell dies and is naturally replaced by the new cells, or can be removed by chemical removers or physical abrasion, or both.

The cosmetic particles contemplated according to the present invention may also comprise an agglomeration of numerous microparticles, such as for example, but not limited to a powder. In this embodiment, the agglomeration of the microparticles is attached to the biofunctional polymer.

In preferred embodiments, the cosmetic product of the present invention is applied to the epidermis of human skin. The epidermis is the topmost layer of the skin that provides waterproofing and serves as a barrier to infection and other external elements. This layer consists mostly of keratinocyte cells, which originate in the basal layer (the deepest layer of the epidermis) from the division of keratinocyte stem cells. The keratinocytes push up through the layers of the epidermis, undergoing gradual differentiation. While these cells move to the surface of the skin the keratinocytes are enucleated, flattened and highly keratinized. Eventually the keratinocytes die off and form the stratum corneum (the outermost layer of the epidermis), which serves as an effective barrier against the entry of foreign matter and infectious agents into the body and minimizes moisture loss. In the stratum corneum, the cell that are each about 10 microns in particle size are stacked tightly and allow for an environment where the pores are small enough to prevent the cosmetic particles from passing through.

In another embodiment, the cosmetic product is applied to hair, such as in the form of a hair coloring and/or conditioner and which may be retained on the hair for an extended duration until it wears off or is manually washed out.

In other embodiments, the cosmetic product of the present invention may be applied nails such as in the form of a polish.

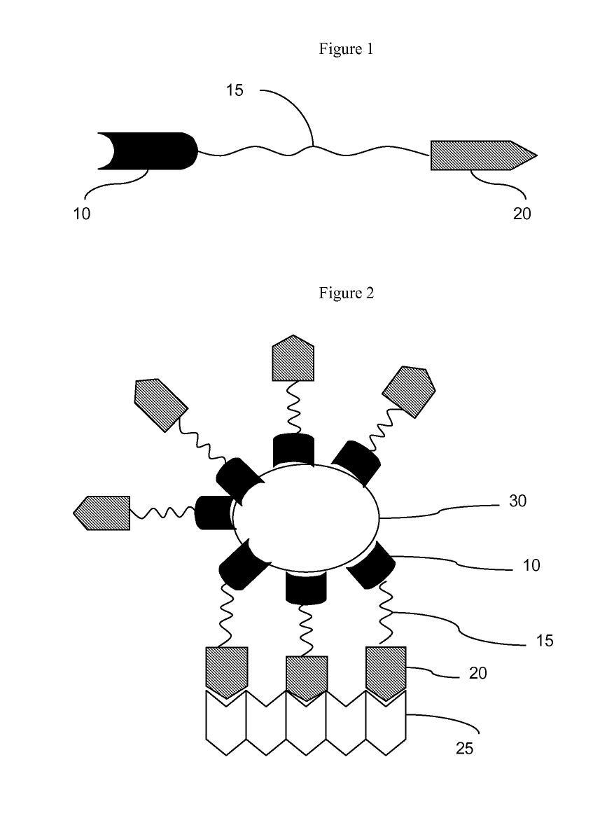

The invention is schematically illustrated in FIGS. 1 and 2. FIG. 1 illustrates a biofunctional polymer unit comprising a particle adsorption unit 10, a biopolymer spacer element 15 as described herein, and a skin (or other biological surface) anchoring biofunctionality component 20. The particle adsorption element is any suitable chemical or physical bond that permits the biofunctional polymer unit to adhere to the particle, for example a silane coupling, covalent bonds, avidin-biotin conjugated complexes, hydrogen bonds, and the like. The skin anchoring biofunctionality component includes peptides, fatty acyl peptides, and the like, as herein described.

FIG. 2 illustrates a particle-biofunctional polymer conjugate of the present invention adhered to a biological surface. The conjugate comprises particle 30, e.g., an inorganic pigment particle, the adsorption element 10, a spacer moiety 15, and a biological surface anchoring biofunctionality component 20, the component 20 being shown adhered to skin surface 25 through a “lock and key” mechanism.

The particles contemplated in accordance with the present invention include, but are not limited to, inorganic colorants, pigments, and optical agents such as soft focus particles to provide the aesthetic aspects of the cosmetic to the skin, hair or nails. There is essentially no limit in the selection of the colorant or pigment. Suitable colorants, including pigments, pearlescent agents, lakes, and dyes, are well known in the art and are disclosed in the International Cosmetic Ingredient Dictionary and Handbook, 11^{th} Edition, 2006 (INCI), the contents or which are hereby incorporated by reference. Exemplary inorganic pigments include, but are not limited to, metal oxides and metal hydroxides such as magnesium oxide, magnesium hydroxide, calcium oxide, calcium hydroxides, aluminum oxide, aluminum hydroxide, iron oxides (α-Fe₂O₃, β-Fe₂O₃, Fe₃O₄, FeO), red iron oxide, yellow iron oxide, black iron oxide, iron hydroxides, titanium dioxide, titanium lower oxides, zirconium oxides, chromium oxides, chromium hydroxides, manganese oxides, cobalt oxides, cerium oxides, nickel oxides, and zinc oxides. Also suitable are composite oxides and composite hydroxides such as iron titanate, cobalt titanate and cobalt aluminate. Other suitable colorants include ultramarine blue (i.e., sodium aluminum silicate containing sulfur), Prussian blue, manganese violet, bismuth oxychloride, talc, mica, sericite, magnesium carbonate, calcium carbonate, magnesium silicate, aluminum magnesium silicate, silica, titanated mica, iron oxide titanated mica, boron nitride, and the like. The colorants may be surface modified with, for example, fluoropolymers, to adjust one or more characteristics of the colorant as described in, for example, U.S. Pat. Nos. 6,471,950, 5,482,547, and 4,832,944, the contents of which are hereby incorporated by reference. Suitable pearling pigments include without limitation bismuth oxychloride, guanine and titanium composite materials containing, as a titanium component, titanium dioxide, titanium lower oxides or titanium oxynitride, as disclosed in U.S. Pat. No. 5,340,569, the contents of which are hereby incorporated by reference. The colorants may also include glittering agents.

Additional colorant/powder fillers include, but are not limited to, inorganic powders such as gums, chalk, Fuller's earth, kaolin, sericite, muscovite, phlogopite, synthetic mica, lepidolite, biotite, lithia mica, vermiculite, aluminum silicate, starch, alkyl and/or trialkyl aryl ammonium smectites, chemically modified magnesium aluminum silicate, organically modified montmorillonite clay, hydrated aluminum silicate, fumed aluminum starch octenyl succinate barium silicate, calcium silicate, magnesium silicate, strontium silicate, metal tungstate, magnesium, silica alumina, zeolite, barium sulfate, calcined calcium sulfate (calcined gypsum), calcium phosphate, fluorine apatite, hydroxyapatite, ceramic powder, metallic soap (zinc stearate, magnesium stearate, zinc myristate, calcium palmitate, and aluminum stearate), colloidal silicone dioxide, and boron nitride; organic powder such as polyamide resin powder (nylon powder), cyclodextrin, methyl polymethacrylate powder, copolymer powder of styrene and acrylic acid, benzoguanamine resin powder, poly(ethylene tetrafluoride) powder, and carboxyvinyl polymer, cellulose powder such as hydroxyethyl cellulose and sodium carboxymethyl cellulose, ethylene glycol monostearate; inorganic white pigments such as magnesium oxide; and stabilizers/rheology modifiers, for example, Bentone Gel and Rheopearl TT2. Other useful powders are disclosed in U.S. Pat. No. 5,688,831, the disclosure of which is hereby incorporated by reference.

The compositions of the invention may optionally comprise functional and/or active and inactive ingredients typically associated with cosmetic and personal care products, including, but not limited to, excipients, emulsifying agents, antioxidants, surfactants, film formers, chelating agents, gelling agents, thickeners, emollients, humectants, moisturizers, vitamins, sodium ascorbyl/cholesteryl phosphate, minerals, viscosity and/or rheology modifiers, sunscreens, UV absorbers, UV blockers, keratolytics, depigmenting agents, retinoids, hormonal compounds, alpha-hydroxy acids, trioxaundecanedioic acid, alpha-keto acids, anti-mycobacterial agents, antifungal agents, antimicrobials, antivirals, analgesics, lipidic compounds, anti-allergenic agents, H1 or H2 antihistamines, anti-inflammatory agents, anti-irritants, antineoplastics, immune system boosting agents, immune system suppressing agents, anti-acne agents, anesthetics, antiseptics, insect repellents, skin cooling compounds, skin protectants, skin penetration enhancers, antiperspirants, exfollients, lubricants, moisturizers, conditioners, fragrances, staining agents, depigmenting agents, hypopigmenting agents, tooth whiteners, preservatives, stabilizers, pharmaceutical agents, photostabilizing agents, and mixtures thereof.

The cosmetic composition contemplated by the present invention can comprise a powder. A cosmetic powder may comprise various fillers and/or additional components and functional agents. Suitable fillers include without limitation silica, treated silica, talc, zinc stearate, mica, kaolin, Nylon powders such as Orgasol™, polyethylene powder, Teflon™, starch, boron nitride, copolymer microspheres such as Expancel™ (Nobel Industries), Polytrap™ (Dow Corning), silicone resin microbeads (Tospearl™ from Toshiba), and polystyrene microspheres, and the like.

The biofunctional polymers include, but are not limited to, peptides, amine groups, amino acids and combinations thereof, along with any other cell-adhering or membrane anchoring biofunctional polymers and compounds that have the functional property of penetrating and attaching to the skin, hair and nails (or any other biological surface) via bonding, adhering, and/or penetration of cells on these keratinous surfaces.

Other biofunctional polymers contemplated by the present invention include cholesterol, cell adhering proteins and peptides, and/or fatty acids. The biofunctional polymer may also comprise a combination of two or more different functional molecules. The amine functional molecule may include R—NH₂, R₁R₂—NH, or R₁R₂R₃N, wherein the length of at least one R group is at least 3 carbon units, and preferable more than 10 carbon units. In addition to the molecular weight and hydrophobicity that determines the number of layers the peptide will penetrate, the biofunctional polymer can alternatively be combined with a spacer of variable length that can also allow for the distance between the surface of the particle and the tip of the peptide.

The peptides utilized contemplated according to the present invention may also include custom designed peptides selected from the group consisting of:

Peptide 1:
NH₂-Cys-Asp-Pro-Gly-Tyr-Ile-Gly-Ser-Arg-Lys-Lys-NH₂, Laminin-B
derived;
Peptide 2:
NH₂-Leu-Arg-Gly-Glu-Val-Gly-Leu-Pro-Gly-Val-Lys-Gly-Asp-Lys-
COOH;
Peptide 3:
NH₂-Gly-Pro-Lys-Gly-Asp-Arg-Gly-Phe-Pro-Gly-Thr-Pro-Gly-Ile-
COOH, Collagen 15 derived;
Peptide 4:
NH₂-Gly-Arg-Gly-Asp-Ser-Pro-Lys-Lys-COOH, Fibronectin derived;
Peptide 5:
NH₂-Lys-Lys-Val-Ala-Ala-Lys-Ser-Gly-Gly-O-Cholesterol, Hedgehog
derived;
Peptide 6:
Fatty acyl-Gly-Arg-Gly-Asp-Ser-Pro-His-Ser-Arg-Asn-COOH;
Peptide 7:
Poly-Arg;
Peptide 8:
Ala-Lys-Pro-Ser-Tyr-Pro-Pro-Thr-(DOPA-Lys-Lys-Lys (natural glue);
Peptide 9:
hybrids of peptide 4 + peptide 2 or peptide 3;
Peptide 10:
Palmitylated/Myristylated/steroylated peptides 1-;
Peptide 11:
D-Phe-Gly-Gly-Gly-Gly-Gly;
Peptide 12:
D-Phe-Phe-Gly-Gly-Gly-Gly;
Peptide 13:
D-Phe-Phe-Phe-Gly-Gly-Gly;
Peptide 14:
D-Phe-Phe-Phe-Phe-Gly-Gly;
Peptide 15:
D-Phe-Phe-Phe-Phe-Phe-Gly;
Peptide 16:
(Phe)ₙ-(Gly)ₘ, where n + m = 1-100;
Peptide 17:
P20;
Peptide 18:
WLR-P20;
Peptide 19:
Ala-Cys-Ser-Ser-Ser-Pro-Ser-Lys-His-Cys-Gly.

Additional peptides that are contemplated and may be used in accordance with the present invention include those peptides identified in Nature Biotechnology, vol. 24, N4, p 455, which is incorporated by reference herein in its entirety.

The peptides may be biotinylated to facilitate conjugation with Avidin-coated microspheres.

The present invention also contemplates the incorporation of a protein transduction domain (“PTD”). As used herein, PTD is intended to comprise peptides are used to enhance cellular uptake of drugs, proteins, polynucleotides or liposomes. PTDs can be fused, attached and/or bonded to other macromolecules, peptides or proteins to successfully transport them into a cell. PTDs may also comprise individual or blended peptides. PTDs may also be blended with biofunctional pigments or attached or fused directly onto pigment particles to enhance the long lasting behavior of pigments.

PTDs contemplated according to the present invention may include, but are not limited to, domains having the following sequences:

Tyr-Ala-Arg-Ala-Ala-Ala-Arg-Gln-Ala-Arg-Ala
Try-Leu-Arg-Arg-Ile-Lys-Ala-Try-Leu-Arg-Arg-Ile-Lys-Ala-Try-Leu-Arg-Arg-
Ile-Lys-Ala

Those of skill in the art will recognize that other PTDs may be used without detracting from the present invention.

The cosmetic composition may also include at least one penetration enhancing compound such as, but not limited to, terpenes, phospholipids, diaglyceride, surfactants, fatty acids, fatty esters, Azone-like compounds, PTDs as described, above peptides such as peptide 19 and others identified in Nature Biotechnology, vol. 24, N4, p 455, which is incorporated by reference herein. The penetration enhancing compound will advantageously be bound to the cosmetic particle and/or the polymer-particle conjugate via bonds including covalent, ionic, hydrogen, as well as other methods of conjugation known to those of ordinary skill in the art.

The selection of a specific biofunctional polymer is determined by the biomolecular target (typically a specific cell or cell type) or the area of the skin or other keratinous surface where the cosmetic particle is to be applied.

The biofunctional polymer can be attached to the particle via a number of different methods: conjugation of Avidin-Biotin complex, siliane coupling covalent bonds, chemisorption, etc. When attached with a biofunctional polymer according to the present invention and applied on skin, the particle sits on the surface of the skin, hair or nail while the polymer adheres to and penetrates specific target cells on the surface to which it is applied resulting in the pigment being anchored and exhibiting long/extended wear.

Preferred coupling agents include silane coupling agents, polar groups or conjugated amines. Examples of silane coupling agents include gamma-(2-aminoethyl)aminopropyltrimethoxysilane, gamma-aminopropyltrimethoxy silane, gamma-amino propyltriethoxysilane, gamma-(2-aminoethyl)aminopropylmethyldimethoxy silane, gamma-methacyryloxypropyltrimethoxy silane, gamma-glycidoxypropyl trimethoxy silane, gamma-mercaptopropyltrimethoxy silane, vinyltriacetoxysilane, gamma-chloropropyltrimethoxysilane, vinyltrimethoxysilane, octadecyldimethyl-[3-(trimethoxysilyl)-propyl]ammonium chloride, gamma-mercaptopropylmethyldimethoxy silane, methyltrichlorosilane, dimethyldichlorosilane, trimethylchlorosilane, gamma-isocyanatopropyltriethoxy silane and the like, and combinations thereof.

The biofunctional particle may also be conjugated with the particle via a spacer molecule. In this manner, the biofunctional polymer-spacer-particle complex is analogous to a lock-and-key principal that provides the biofunctionality to an inorganic pigment that can not penetrate in the skin cell because of its selective molecular size and surface energy. Within this lock-and-key, an adsorption element in the polymer attaches to the inorganic cosmetic particle that is connected to biopolymer spacer and allows the biofunctional molecule such as a peptide to be linked to the cosmetic particle.

Spacer molecules are known in the art. The invention is not limited, however, in the selection of a spacer except that it is up to the skilled practitioner to select a spacer that will conjugate with the desired particle and biofunctional polymer for adhering to specific cells on a keratinous surface. Additionally, the spacer may be a peptide composed of any amino acid and mixtures thereof. For example, the peptide spacers may be composed of the amino acids glycine, alanine, and serine, and mixtures thereof. The peptide spacers may be linked to the binding peptide sequence by any method known in the art. For example, the binding peptide-spacer-particle complex may be prepared using standard peptide synthesis methods known in the art. The spacer may also be a combination of a peptide spacer and an organic spacer molecule.

In preferred embodiments, the particle is formulated in a basic benign solvent (i.e., water) without the presence of harsh solvents and/or polymers that result in undesired attributes such as skin tightening, drying, and other undesirable aesthetic and tactile properties. As a result of the anchoring of the particle to the skin via the use of a biofunctional polymer according to the invention, the particles are protected from being washed off the skin with water or being removed by normal physical abrasion. The particles anchored to the skin via the biofunctional polymer are unaffected by sweat, sebum or any other chemicals introduced on the skin via the environment or from other products applied to the skin that typically compromise long wear and aesthetics.

The biofunctional polymer particle of the present invention will help retain the cosmetic particles on the skin (or other keratinous surface) for a long period of time without the need for harsh solvents or polymers that have negative feel attributes.

The duration of wear of the cosmetic particle on the skin depends on the selected biofunctional polymer. Specific biofunctional polymers may be selected such that the cosmetic particle remains anchored on skin until the cell to which it is attached dies and is naturally replaced by new skin. Alternatively, if desired, the cosmetic particle may be coated with a biofunctional polymer that can remain until it is removed with a commercial cosmetic remover or via physical abrasion or both.

The cosmetic product of the invention will typically have the form of powders, hot-pours and/or emulsions for application upon the lips, face and/or eyes. While it is the primary intention of the present invention to be used primarily in color cosmetic products for long wear and that have the form of powders, hot-pours, and/or emulsion forms for use in lip, face and/or eye products, it is also contemplated that the invention may be used for different surfaces such as hair to introduce a non-dying hair color system where the hair color can remain for multiple days because of bio-functional polymers without the use of harsh solvents and chemical reactions. Other surfaces such as nails and teeth may find a benefit through longer-lasting color in a nail polish product and a longer-lasting teeth whitening system that may contain actives that remain on teeth for long periods of time. Similarly, this invention can also retain powder fragrances on a substrate for longer periods of time. Actives such as deodorants, sunscreens, insect repellants and the like, can find huge benefit from this biofunctional long-wearing active particle delivery system, as well maintaining their benefit for longer/sustained periods without the trade-offs of incorporated harsh solvents and polymers.

In connection with formulations for nail products that encompass the biofunctional polymer and a particle according to the instant invention, it is intended that such nail products will be nitrocellulose-free and acetate and acetone-free. Nitrocellulose is one of the primary ingredients in nail polish is both flammable and explosive. Nitrocellulose acts as a film forming agent. For nail polish to work properly, a hard film must form on the exposed surface of the nail, but it cannot form so quickly that it prevents the material underneath from drying. By itself or used with other functional ingredients, the nitrocellulose film is brittle and adheres poorly to nails. In accordance with the present invention, a colorant or color pigment will be coated with a biofunctional polymer that facilitates long-term adherence of the color to nails and, in turn, eliminates the dangerous nitrocellulose ingredient from the commercial product.

Furthermore, nail polishes in accordance with the present invention are contemplated without the inclusion of acetate and/or acetone solvents. Acetone (propanone) is harmful to the environment and flammable. Removal of these dangerous solvents is highly desirable.

All percentages are by weight, based on the total weight of the composition, unless otherwise indicated.

All references including patent applications and publications cited herein are incorporated herein by reference in their entirety and for all purposes to the same extent as if each individual publication or patent or patent application was specifically and individually indicated to be incorporated by reference in its entirety for all purposes.

Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

1. An extended wear cosmetic composition for application to a keratinous surface comprising a cosmetic particle coated with a biofunctional polymer having amino or amino acid functionality wherein said coated particle adheres to the keratinous surface for an extended time period.

2. The cosmetic composition according to claim 1 wherein the keratinous surface is selected from skin, hair or nails.

3. The cosmetic composition according to claim 1, wherein the cosmetic particle comprises colorants and pigments.

4. The cosmetic composition according to claim 1 wherein the cosmetic particle has a median particle size from about 2 microns to less than about 50 microns.

5. The cosmetic composition according to claim 1, wherein said cosmetic particle is Avidin-coated.

6. The cosmetic composition according to claim 1 wherein said amino or amino acid functionality is a peptide selected from the group consisting of:
a.
NH₂-Cys-Asp-Pro-Gly-Tyr-Ile-Gly-Ser-Arg-Lys-Lys-NH₂, Laminin-B derived;
b.
NH₂-Leu-Arg-Gly-Glu-Val-Gly-Leu-Pro-Gly-Val-Lys-Gly-Asp-Lys-COOH;
c.
NH₂-Gly-Pro-Lys-Gly-Asp-Arg-Gly-Phe-Pro-Gly-Thr-Pro-Gly-Ile-COOH,
Collagen 15 derived;
d.
NH₂-Gly-Arg-Gly-Asp-Ser-Pro-Lys-Lys-COOH, Fibronectin derived;
e.
NH₂-Lys-Lys-Val-Ala-Ala-Lys-Ser-Gly-Gly-O-Cholesterol, Hedgehog derived;
f.
Fatty acyl-Gly-Arg-Gly-Asp-Ser-Pro-His-Ser-Arg-Asn-COOH;
g.
Poly-Arg;
h.
Ala-Lys-Pro-Ser-Tyr-Pro-Pro-Thr-(DOPA-Lys-Lys-Lys (natural glue);
i.
Hybrids of peptide 4 + peptide 2 or peptide 3;
j.
Palmitylated/Myristylated/steroylated peptides 1-;
k.
D-Phe-Gly-Gly-Gly-Gly-Gly;
l.
D-Phe-Phe-Gly-Gly-Gly-Gly;
m.
D-Phe-Phe-Phe-Gly-Gly-Gly;
n.
D-Phe-Phe-Phe-Phe-Gly-Gly;
o.
D-Phe-Phe-Phe-Phe-Phe-Gly;
p.
(Phe)ₙ-(Gly)ₘ, where n + m = 1-100;
q.
P20;
r.
WLR-P20;
s.
Ala-Cys-Ser-Ser-Ser-Pro-Ser-Lys-His-Cys-Gly, and combinations thereof

7. The cosmetic composition according to claim 6 wherein said peptides are biotinylated to facilitate conjugation with Avidin-coated particles.

8. The composition according to claim 1 further comprising a protein transduction domain.

9. The composition according to claim 8 wherein the protein transduction domain is covalently attached to the biofunctional polymer.

10. The cosmetic composition according to claim 1 wherein said functionality includes R—NH₂, R₁R₂—NH, or R₁R₂R₃N, wherein the length of at least one R group is greater than 5 carbon units.

11. The cosmetic composition according to claim 1 further comprising a coupling agent.

12. The cosmetic composition according to claim 11 wherein the coupling agent is selected from the group consisting of silane coupling agents, polar groups, and conjugated amines.

13. The cosmetic composition according to claim 12 wherein the coupling agent is a silane coupling agent.

14. The cosmetic composition according to claim 13 wherein the silane coupling agent is selected from the group consisting of gamma-(2-aminoethyl)aminopropyltrimethoxysilane, gamma-aminopropyltrimethoxy silane, gamma-aminopropyltriethoxysilane, gamma-(2-aminoethyl)amino propylmethyldimethoxy silane, gamma-methacyryloxypropyltrimethoxy silane, gamma-glycidoxypropyltrimethoxysilane, gamma-mercapto propyltrimethoxy silane, vinyltriacetoxysilane, gamma-chloro propyltrimethoxysilane, vinyltrimethoxysilane, octadecyldimethyl-[3-(trimethoxysilyl)-propyl]ammonium chloride, gamma-mercapto propylmethyldimethoxy silane, methyltrichlorosilane, dimethyldichlorosilane, trimethylchlorosilane, gamma-isocyanatopropyltriethoxy silane and the like, and combinations thereof.

15. The cosmetic composition according to claim 1 further comprising a biomolecular spacer that conjugates said particle with said biofunctional polymer.

16. The cosmetic composition according to claim 1 further comprising at least one penetration enhancing compound to enhance penetration of the amino or amino acid functionality through said keratinous surface.

17. The cosmetic composition according to claim 16 wherein the penetration enhancing compound may be selected from the group consisting of terpenes, phospholipids, phosphosphingolipids, diacylglyceride, surfactants, fatty acids, fatty esters, and Azone-like compounds and protein transduction domains.

18. The cosmetic composition according to claim 17 wherein the penetration enhancing compound is bonded to the biofunctional polymer.

19. A delivery system comprising a particle and a biofunctional polymer having amino or amino acid functionality coated on or conjugated with the particle, wherein the particle adheres to a keratinous surface for an extended time period.

20. The delivery system according to claim 19 wherein the biofunctional polymer has an affinity for specific cells on said keratinous surface.

21. The delivery system according to claim 19 wherein said particle is a cosmetic particle, a biologically active agent and/or an optical agent.

22. The delivery system according to claim 19 wherein said amino or amino acid functionality is a peptide selected from the group consisting of:
a.
NH₂-Cys-Asp-Pro-Gly-Tyr-Ile-Gly-Ser-Arg-Lys-Lys-NH₂, Laminin-B derived;
b.
NH₂-Leu-Arg-Gly-Glu-Val-Gly-Leu-Pro-Gly-Val-Lys-Gly-Asp-Lys-COOH;
c.
NH₂-Gly-Pro-Lys-Gly-Asp-Arg-Gly-Phe-Pro-Gly-Thr-Pro-Gly-Ile-COOH,
Collagen 15 derived;
d.
NH₂-Gly-Arg-Gly-Asp-Ser-Pro-Lys-Lys-COOH, Fibronectin derived;
e.
NH₂-Lys-Lys-Val-Ala-Ala-Lys-Ser-Gly-Gly-O-Cholesterol, Hedgehog derived;
f.
Fatty acyl-Gly-Arg-Gly-Asp-Ser-Pro-His-Ser-Arg-Asn-COOH;
g.
Poly-Arg;
h.
Ala-Lys-Pro-Ser-Tyr-Pro-Pro-Thr-(DOPA-Lys-Lys-Lys (natural glue);
i.
Hybrids of peptide 4 + peptide 2 or peptide 3;
j.
Palmitylated/Myristylated/steroylated peptides 1-;
k.
D-Phe-Gly-Gly-Gly-Gly-Gly;
l.
D-Phe-Phe-Gly-Gly-Gly-Gly;
m.
D-Phe-Phe-Phe-Gly-Gly-Gly;
n.
D-Phe-Phe-Phe-Phe-Gly-Gly;
o.
D-Phe-Phe-Phe-Phe-Phe-Gly;
p.
(Phe)ₙ-(Gly)ₘ, where n + m = 1-100;
q.
P20;
r.
WLR-P20;
s.
Ala-Cys-Ser-Ser-Ser-Pro-Ser-Lys-His-Cys-Gly, and combinations thereof

23. The delivery system according to claim 19 further comprising a coupling agent.

24. The delivery system according to claim 19 further comprising a biomolecular spacer that conjugates said particle with said biofunctional polymer.
